(19) United States
(12) Patent Application Publication
(10) Pub. No.: US 2018/0099064 A1
(43) Pub. Date: Apr. 12, 2018

(54) FRAGRANCE SACHET

(71) Applicant: The Willowbrook Company, LLC, Spartanburg, SC (US)

(72) Inventors: Christopher Burns, Boiling Springs, SC (US); Heidi S. Ruehl, Spartanburg, SC (US)

(21) Appl. No.: 15/723,354

(22) Filed: Oct. 3, 2017

Related U.S. Application Data
(60) Provisional application No. 62/405,079, filed on Oct. 6, 2016.

(51) Int. Cl. A61L 9/12; A61L 9/04

(52) U.S. Cl. CPC A61L 9/12; A61L 9/046

(57) ABSTRACT

A fragrance sachet includes a holder for a medium such as vermiculite. The medium can be treated with fragrance materials such as essential oils. The holder comprises a pouch formed with different materials on opposing sides which form designated respective front and back of the sachet. The designated front material is selected to support the application of indicia and/or decorations, while the designated back material may have filter type characteristics, to facilitate fragrance dispersion. The use of heat-sealable opposing side materials particularly facilitates formation of the pouch using a form-fill-seal system. The resulting sachet product may be configured with self-standing and/or hanging support features which avoid damaging any associated finished surfaces.

### PRIORITY CLAIM

This application claims the benefit of previously filed U.S. Provisional Patent Application entitled “FRAGRANCE SACHET,” assigned U.S. Ser. No. 62/405,079, filed Oct. 6, 2016, and which is incorporated herein by reference for all purposes.

### FIELD OF THE SUBJECT MATTER

The presently disclosed subject matter relates generally to devices for disseminating fragrance, without requiring power.

### BACKGROUND OF THE SUBJECT MATTER

It is generally desirable to be able to conveniently disseminate a desired fragrance in a designated target area. Fragrance sachets have been provided which comprise paper envelopes, which are glue-sealed enclosures holding scent-impregnated materials. Over time, the nature of the paper or other components such as separately applied adhesives can either naturally degrade or be caused to degrade through direct contact or close association with fragrance materials and/or vapors from such fragrance materials, thereby limiting the life of a particular product.

Furthermore, we have found that certain materials, while useful for allowing fragrance to diffuse into the room or target area also allow the fragrance oils to damage finished surfaces.

Accordingly, an improved fragrance sachet which permits the sachet to be reliably constructed while formed in a variety of shapes, and/or a variety of materials and/or product shapes and features that offer protection to contiguous surfaces, and/or with a variety of decorations applied thereon would be useful.

### BRIEF DESCRIPTION OF THE SUBJECT MATTER

The presently disclosed subject matter recognizes and variously addresses the foregoing issues, and others concerning certain aspects of fragrance sachets. Thus, broadly speaking, an object of certain embodiments of the presently disclosed subject matter is to provide improved designs for fragrance sachets.

More particularly, one exemplary embodiment of a presently disclosed fragrance sachet includes a holder for a medium such as vermiculite, sawdust, zeolite, shredded paper, or something similar. The medium can be treated with fragrance materials such as essential oils, to impart a selected fragrance. The holder may comprise a formed pouch with different materials forming the front and back. The front material may be, for one example, a foil-based substrate (or generally not vapor permeable material), to support the application of indicia and/or decorations, while the back material may be, for one example, a filter type material (or generally at least partially vapor permeable material), to facilitate fragrance dispersion. The front and back materials may be heat-sealable, to facilitate the formation of the pouch, or alternatively, the materials may be fused using ultrasonic bonding, stapled, separately applied adhesives, crimped, or other methods.

A second exemplary embodiment of a presently disclosed fragrance sachet includes a holder for a medium such as vermiculite, sawdust, zeolite, shredded paper, or something similar. The medium can be treated with fragrance materials such as essential oils, to impart a selected fragrance. The holder may comprise a formed pouch with different materials forming the front and back. For instance, if a finished surface is closest to the back of the device, then the back material may be, for one example, a foil-based, or polymeric film substrate (or generally not vapor permeable material), to protect the surface against the ingress of fragrance oils. The front material may be, for a second example, a filter type material (or generally at least partially vapor permeable material such as a plant based or synthetic paper, or a non-woven, a woven, or a knitted material), to facilitate fragrance dispersion while providing support to the optional application of indicia and/or decorations. The front and back materials may be heat-sealable to facilitate the formation of the pouch, or alternatively, the two materials may be fused using ultrasonic bonding, stapled, separately applied adhesives, crimped, or other methods.

Additional aspects and advantages of the present disclosed subject matter relate to the protection of adjacent hard finished surfaces from the deleterious effects of high concentrations of perfume oil. Such surfaces include polished and/or varnished wood, polyurethane coated leather or upholstery, artificial leather, leather, silk, painted wood, powder coated metal, lacquered surfaces, plastic decorations such as those made from methylmethacrylates, such as Lucite or Perspex brand man-made PMMA materials, or acrylics.

Another exemplary embodiment of presently disclosed subject matter relates to an improved fragrance sachet, comprising a holder comprising a pouch formed of joined opposing sections, such joined opposing sections comprising respective different materials; and a fragranced medium received within such pouch, for imparting a selected fragrance from such fragrance sachet.

Yet another presently disclosed subject matter exemplary embodiment relates to a fragrance sachet device for disseminating fragrance, without requiring power. Such a device preferably comprises a holder for a medium treated with essential oils, such holder comprising a pouch formed with respective different at least first and second materials on opposing sides thereof, with one of such materials selected from a vapor impermeable material to protect a supporting surface against the ingress of essential oils, and the other of such materials selected from a filter type material to facilitate fragrance dispersion.

Still another exemplary embodiment of presently disclosed subject matter relates to a multiple-component, self-supporting, pod-based fragrance sachet, comprising a supporting base structure; a pouch; a fragranced medium received within such pouch; and a pod cover piece for covering such pouch, and interfacing with such supporting base structure for support of such fragrance sachet.

It should be understood from the complete disclosure herewith that the presently disclosed subject matter equally relates to corresponding and/or related methodology.

In yet a further exemplary embodiment of the presently disclosed subject matter, methods for making the presently disclosed fragrance sachets are provided.

Another presently disclosed exemplary methodology relates to methodology for producing a form/fill/seal fragrance sachet, comprising providing a single sheet of at least partially vapor permeable material; treating peripheral regions around the entirety of such single sheet of material; folding such single sheet of material about a symmetrical foldline so that respective edges make contact whenever such sheet is folded; sealing at selected respective peripheral regions so that a resulting pouch is formed with an open mouth; filling such pouch with a fragranced medium; and sealing the open mouth of such resulting pouch, to provide a formed/filled/sealed fragrance sachet.

Yet further exemplary methodology relates to methodology for providing a multiple-component, self-supporting, pod-based fragrance sachet, comprising: providing a supporting base structure; providing a pouch with a fragranced medium received within such pouch; and providing a pod cover piece for covering such pouch, and interfacing with such supporting base structure for support of such fragrance sachet.

Still further, another exemplary method according to presently disclosed subject matter relates to methodology for producing a fragrance sachet, comprising the steps of: sealing a vapor impermeable-based substrate to a filter-based substrate to form a partially open pouch; filling the pouch with scent-impregnated material; and closing the pouch to form a fragrance sachet.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features and elements hereof may be practiced in various embodiments and uses of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the Figures or stated in the detailed description of such Figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification.

### BRIEF DESCRIPTION OF THE DRAWINGS

A full and enabling disclosure of the presently disclosed subject matter, including the best mode thereof, directed to one of ordinary skill in the art, is set forth in the specification, which makes reference to the appended Figures, in which:

FIG. 1 provides a front perspective view of a fragrance sachet according to an exemplary embodiment of the presently disclosed subject matter;

FIG. 2 provides a rear perspective view of the exemplary fragrance sachet of FIG. 1;

FIG. 3 provides a front perspective view of another exemplary embodiment of the presently disclosed subject matter;

FIGS. 4A through 4D provide respective front perspective views of four additional exemplary embodiments of the presently disclosed subject matter, with respective integrated or associated attachment or mounting/hanging features or means;

FIGS. 5A and 5B provide respective front perspective views of two additional exemplary embodiments of the presently disclosed subject matter, with respective integrated or associated attachment or mounting/hanging features or means;

FIG. 6 provides a generally side edge view of another exemplary embodiment of the presently disclosed subject matter incorporating or associated with alternative attachment or mounting features or means;

FIGS. 7A through 7C provide respective side edge, and opposing side views of another exemplary embodiment of the presently disclosed subject matter, incorporating an exemplary alternative resulting pouch shape;

FIG. 8 is a front perspective view of another exemplary embodiment of the presently disclosed subject matter, illustrating another exemplary alternative resulting pouch shape;

FIGS. 9A and 9B illustrate partially separated and partially assembled configurations, respectively, of a pod-based exemplary embodiment of the presently disclosed subject matter, for alternative self-supporting features or means thereof;

FIG. 10 is a generally side elevation view of an exemplary pod feature of the presently disclosed subject matter for use in accordance with the exemplary embodiment represented in application FIGS. 9A and 9B;

FIGS. 11A through 11D represent steps in an exemplary presently disclosed methodology for formation of a pod feature for use in accordance with the exemplary embodiment represented in application FIGS. 9A and 9B; and

FIGS. 12A and 12B illustrate top elevation drawings representative of an exemplary paper coating process for a form/fill/seal paper sachet.

Repeat use of reference characters throughout the present specification and appended drawings is intended to represent same or analogous features, elements, or steps of the presently disclosed subject matter.

### DETAILED DESCRIPTION OF THE SUBJECT MATTER

Reference is made herein in detail to embodiments of the presently disclosed subject matter, one or more examples of which are illustrated in the drawings. Each example is provided by way of explanation of the presently disclosed subject matter, and not a limitation of the presently disclosed subject matter. In fact, it will be apparent to those skilled in the art that various modifications and variations can be made in the presently disclosed subject matter without departing from the scope or spirit of the presently disclosed subject matter. For instance, features illustrated or described as part of one embodiment can be used with another embodiment to yield a still further embodiment. Thus, it is intended that the presently disclosed subject matter covers such modifications and variations as come within the scope of the appended claims and their equivalents.

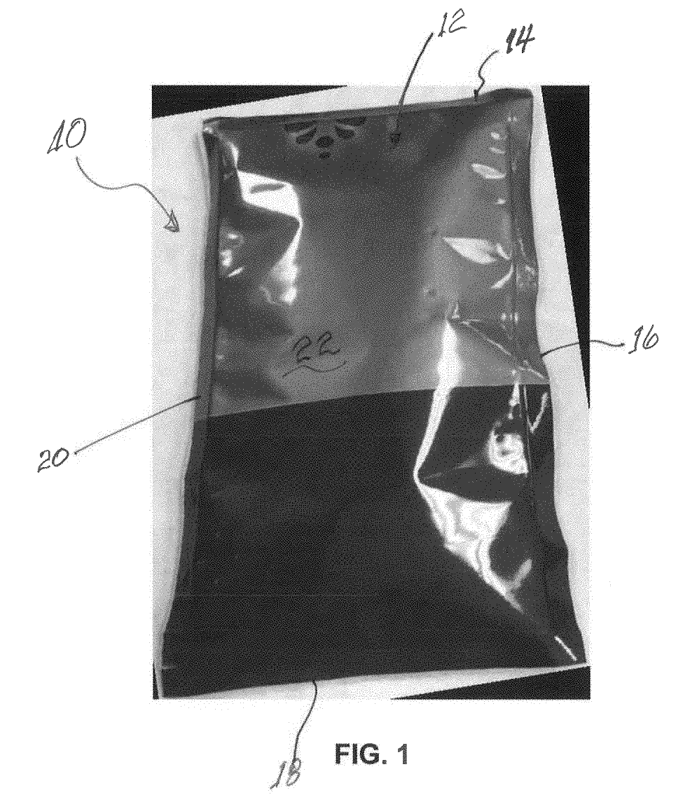

FIG. 1 provides a generally front perspective view of a fragrance sachet generally 10 according to an exemplary embodiment of the presently disclosed subject matter with a foil-based substrate 12 forming a pouch. Such pouch in the illustrated exemplary embodiment is generally rectangular, and four respective side edges generally 14, 16, 18, and 20. As shown, such side edges surround a bulkier, central portion (generally 22) which encloses a fragrance-impregnated medium (not shown).

As always shown in the exemplary embodiment of present FIG. 1, such substrate may support various indicia, in the form of colors or designs.

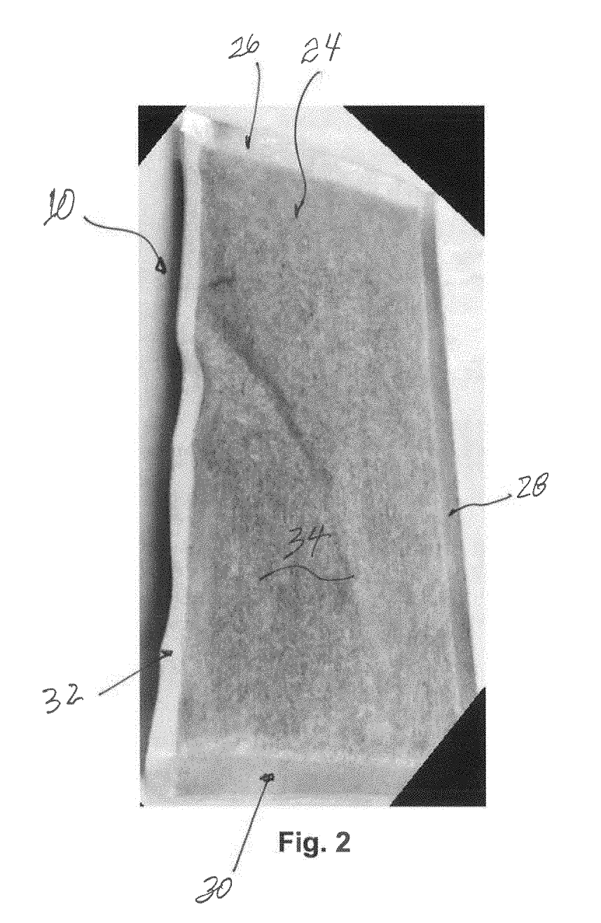

FIG. 2 represents a rear perspective view of the exemplary fragrance sachet generally 10 of application FIG. 1. Instead of a foil-based substrate 12, a filter-based substrate generally 24 is provided. For the exemplary shape shown, it likewise includes four edge portions (26, 28, 30, and 32) around the periphery thereof, and a bulkier central portion generally 34. Together, respective central portions 22 and 34 enclose a scent-impregnated medium (not shown), and are secured by joining of the respective front edge portions with their correspondingly-positioned rear edge portions. Such securement may preferably be by heat-sealing, ultrasonic bonding, crimping, gluing, folding, or stapling, or by other means.

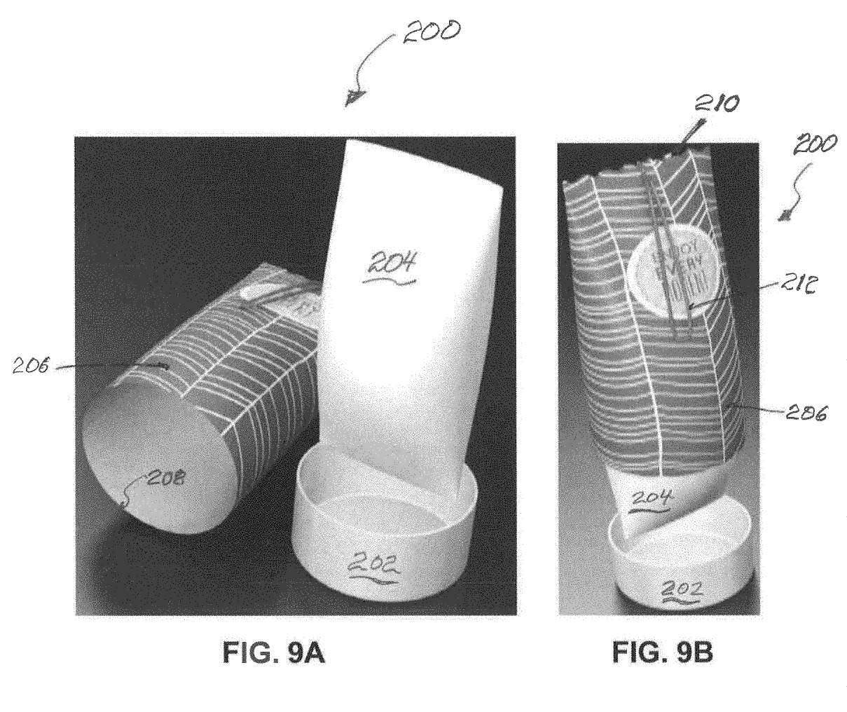

Certain materials, such as water based latexes, PE emulsions or dispersions, may be printed onto the paper layer in specific areas—such as around the perimeter of the sheet. Once dried, these materials may be used to thermally bond the paper to a contiguous thermoplastic film sheet, via thermal, ultrasonic, radiofrequency, or microwave means. An example of such a material is CK-5791-26FDA from Cork Industries Inc. (FL). A second example of such a material is EvCote Waterbarrier 3000, available from Akzo Nobel Coatings Inc. (IN), which is made from recycled PET and plant based oils, and also may be used as an oil barrier coating in food and beverage packaging applications. It is to be understood by those of ordinary skill in the art that such potential coatings for paper is preferably used with pod sachet embodiments, as discussed in conjunction with FIGS. 9A through 11D herein. For presently disclosed exemplary embodiments which are various combinations of filter-type materials and foil, the combined selected materials are preferably already inherently amenable to heat sealing without requiring any additional coatings on the filter paper.

When the foil-based substrate 12 is sealed to the filter-based substrate 24, and filled with scent-impregnated material (not shown), a fragrance sachet is formed.

In other words, various beneficial attributes of the sachet are created by filling and forming two different materials, in this exemplary embodiment, filter and barrier film (or foil), in such a way that the resulting sachet has both an artistic and graphic presentation on the designated front that is a fragrance impervious substrate on the designated front, while the back is a functional/breathable surface that allows diffusion of fragrance from the back. Such impervious substrates may include but are not limited to metal foils, composites containing metal foils, certain metallized polymer films, polyimides such as Kapton® man-made films, polyethylene terephthalate (PET), or high density poly(ethylene), poly(propylene) (PP), oriented poly(propylene) (OPP), poly(acrylonitrile), certain poly(amides) including nylon 6 and nylon 6,6, poly(vinyl fluoride) films such as Tedlar® films, and copolymers such as Barex® resin (a copolymer of (acrylonitrile) and (methyl acrylate) grafted with nitrile rubber), UV cured cross-linked films, films and papers coated with UV cured coatings, thereby protecting adjacent or contiguous surfaces from damage due to high concentrations of fragrance oil. The barrier film may also be laminated composites of various film materials.

The idea of taking a printed and very graphic foil-based material and fusing it to filter-based material that can still release fragrance, and form/fill/sealing it, is a combination which can all be performed with one piece of equipment.

For example, a medium such as vermiculite or similar bulk material which is absorbent may be fragranced, such as with the use of essential oils. Essential oils as referenced herein is intended to comprise any of naturally occurring fragrance oils or synthetic or manufactured blends of fragrance oils, or blends between natural and manufactured origin materials. Other materials such as powders, volcanic rock, pumice, or other possible absorbent materials may be practiced in some embodiments. Filter material may be combined with foil-based material to form a pouch for receiving the fragranced vermiculite.

Foil or film material should be used with suitable properties, for being heat-sealable, crimped, fastened with grommets, stapled, ultrasonic bonded, with a selected filter-based material.

In one exemplary embodiment, a nonwoven polyester based material may be used. For both substrates, the materials 12 and 24 are trimmed to an appropriate size, and then the two sides may be fused, while leaving an opening for introduction of the medium.

In some production arrangements, a machine may be provided which uses two different rolls of material, which are run so as to be positioned appropriately before being fused in part, and then trimmed. Production should be controlled in terms of the amount of heat and the timing of the machine speed, to ensure that neither side receives any heat (burn) damage, while otherwise receiving sufficient heat bonding. Those of ordinary skill in the art will appreciate that such results can be obtained for variously selected combinations of specific foil and filter substrates by adjusting out with temperature and machine speed. As also understood, if fastening by heat sealing or ultrasonic bonding, the substrates would need the proper laminate (a thermoplastic, such as PP, PE, or PET), but otherwise a machine can heat seal the two different types of material in the illustrated, generally rectangular, pillow pack shape.

The resulting combination of foil and filter materials to create a fragrance sachet allows a finished surface for printing (on the designated front), while having a breathable porous surface (on the designated back) for the circulation of scent from the impregnated medium enclosed in the pouch. Alternatively, if protection of an adjacent or proximate surface is desired, then the side closest to the proximate surface (e.g. the back side) may be an impervious film or foil or composite, whereas the front side (distant from the proximate or adjacent surface) may be a decorated porous material that allows fragrance to diffuse into the air. Optionally, a vent clip may be attached to the impervious surface so that the air freshener can be clipped onto an auto or home air conditioner vent. In this way, the air freshener surface closest to the finished automotive interior will prevent fragrance oils from diffusing into the automotive finishes, reducing the chance of surface damage.

Another example of a desired filter material 24 comprises a lightweight material, such as comprised of two-phase heat-sealable tissues comprising a blend of thermoplastic fibers and selected cellulosic fibers. The resulting filter materials provide a high wet strength, and a neutral odor, both of which are benefits for use in the subject fragrance sachet.

Alternatively, the filter material may be printed with a coating that is heat or ultrasonically sealable to another thermoplastic material. However, for many of the preferred embodiments of presently disclosed subject matter, the combined selected materials are preferably already inherently amenable to heat sealing without requiring any additional coatings on the filter paper. In other words, the nature of the filter material itself allows heat sealing to another thermally-sealable material, without requiring any coating or separate treatment of the filter paper component. In instances where a further paper component is heat sealed to a filter paper component, it may in some embodiments be preferred to include a spot coating approach to allow the two materials to be heat sealed with each other, as will be understood by those of ordinary skill in the art.

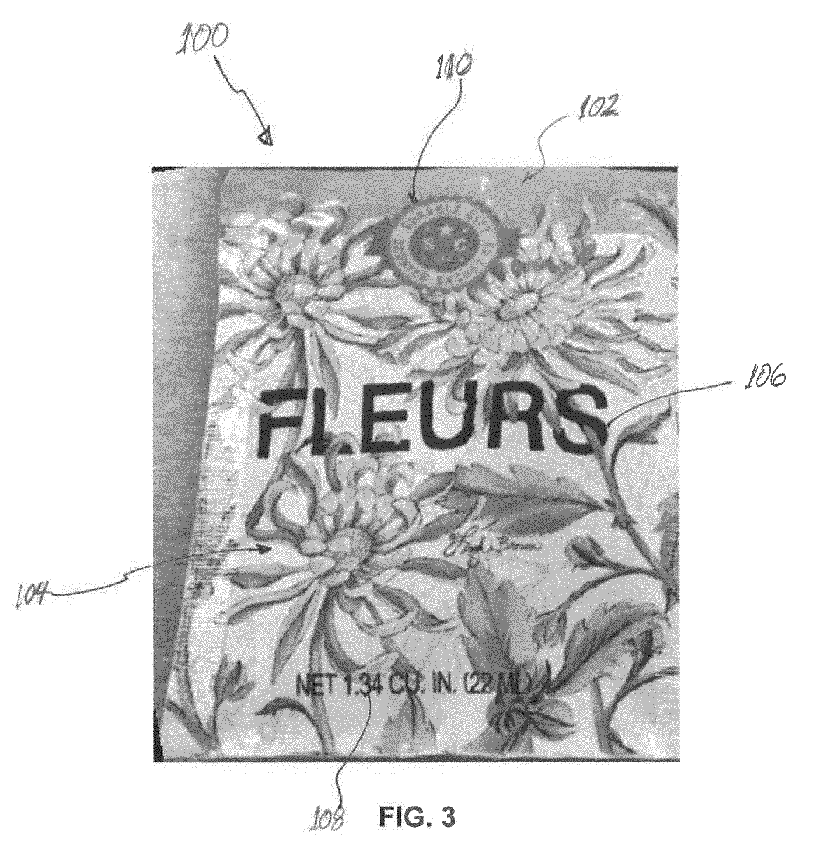

Application FIG. 3 illustrates a generally front perspective view of another exemplary embodiment in accordance with presently disclosed subject matter. A fragrance sachet generally 100 has a foil-based substrate 102, on which may be printed various decorative indicia such as 104, or printed subject matter of an informative or other nature, as represented by indicia 106, 108, and 110. Variations may be practiced. For example, ink or pigment may be applied or printed on the outer surface of substrate 102. As will be understood by those of ordinary skill in the art without limitation, such applied material may define any variety of repeating geometric patterns of quatrefoils, stars, squares, and other geometric or non-geometric shapes (decorative images) printed onto such outer surface of substrate 102. Pigment 336 may be applied or printed onto a surface of the substrate of material prior to heat treatment or other application of such first substrate of material to a second substrate to form a pouch in the manner discussed above.

Certain materials are good for allowing the diffusion of fragrances into the room or another enclosed space such as a car interior. Such materials may include various porosities of papers, non-woven materials, or perforated films. Other film or sheet materials are not very useful for diffusing fragrance, however, still allow the slow migration of fragrance oils over time through the film. These may include vinyl films such as PVC, for instance, or styrene-butadiene copolymers, polyurethane films, latex films. If a sachet made from these materials is left in contact with a finished surface (e.g. varnished wood, painted metal, painted wood, treated wood, plastic composites, finished upholstery and furniture, coated metal, etc.) then, even though the diffusion of the fragrance is slow enough to preclude the material from being used as a diffusion filter, nevertheless, fragrance oil diffuses into the finished surface and may damage it.

Water Porosity Test:

The porosity of film and sheet materials can easily be measured in terms of amount of liquid water that the material can absorb. This test is a proxy for a Sheffield Porosimeter or a Gurley Densometer test that may alternatively be used to test paper porosity. The assumption is that, the more generally absorbent a material is, the more porous that material is to air. The thickness of a sheet of about 4*4 inches of the material to be tested is measured using a Vernier Calliper (Sealey Professional Tools, Suffolk UK) and recorded. An individual sheet of paper is utilized. It is understood that some such samples more typically may be sold in bulk, and rated by bulk weight, for example, with the photocopy paper in this instance rated at 20 pounds per 500 sheets (of 92 brightness paper). The dry mass (equilibrated at room temperature for 2 hours) is also measured. The sheet is immersed in distilled or deionized water at room temperature for up to 5 minutes, then removed.

The sheet is not squeezed, rather it is gently blotted with laboratory tissue paper, such as KimWipes, available from Kimberly-Clark Neenah Wis., to remove visible surface droplets and then immediately weighed. The weight is recorded as Mw.

The water porosity value is calculated by:

$$Pw = \frac{Mw - Md}{Md}$$

Where Pw=water porosity value in g per g

Mw=mass of wet paper in g

Md=mass of dry paper in g

$$TAP = \frac{1000 * Pw}{d}$$

Where TAP=thickness adjusted porosity in g per g per millimeter

And d=thickness of the sheet in microns.

| | | Dry | | | |
|---|---|---|---|---|---|
| Material | Thickness | weight | Wet Wght | Pw | TAP |
| Viva ® | 560 | 1.135 | 10.695 | 8.42 | 15.0409 |
| Paper | | | | | |
| Towel | | | | | |
| Sachet | 140 | 1.209 | 2.845 | 1.35 | 9.6656 |
| Paper | | | | | |
| PP NW | 250 | 0.568 | 3.308 | 4.82 | 19.2958 |
| Wax Paper | 20 | 0.514 | 0.745 | 0.45 | 22.4708 |
| Card | 300 | 1.735 | 2.924 | 0.69 | 2.2843 |
| Blotting | 240 | 0.927 | 5.352 | 4.77 | 19.8894 |
| paper | | | | | |
| Photocopy | 80 | 0.661 | 1.463 | 1.21 | 15.1664 |
| paper | | | | | |
| Tyvek ® | 130 | 0.513 | 0.591 | 0.15 | 1.1696 |
| man-made | | | | | |
| fiber sheets | | | | | |
| Silk | 150 | 0.785 | 2.607 | 2.32 | 15.4735 |
| Charmeuse | | | | | |
| Microfiber | 290 | 0.483 | 1.737 | 2.60 | 8.9527 |
| PET knit. | | | | | |

Pass-Through Test for Gross Porosity

The gross porosity refers to much larger pores or perforations in materials, that allow free fluid movement through the film/fabric/or paper. This property may be tested in the following manner: The thickness of the sheet material is measured using a Vernier Calliper (Sealey Professional Tools, Suffolk UK.) A square 5″×5″ sheet of the material to be tested is folded in half twice, to make a four layer thick square of 2½″×2½″. The top layer of the square is gently opened, and placed corner down into a glass funnel. In this way, a liquid may be added between the first and second sheet and would be retained by the sheet material if it were not grossly porous. 13 ml of water is added to the folded material in the funnel, pouring it all between first and second folded sheet. A stopwatch is started as the first drop of liquid falls into the paper, and stopped when the first drop of liquid forms within the glass funnel.

The Gross Porosity is determined by:

$$GP = \frac{1}{t}$$

Where t=the time for liquid breakthrough in seconds,
And GP is gross porosity of units: per second, (s⁻¹)

The Thickness Adjusted Gross Porosity (TAGP) is determined by:

$$TAGP = \frac{GP * 1000}{d}$$

where d=material thickness in microns
And TAGP has the units of per millimeter per second (mm⁻¹ s⁻¹) and is a measure of how easily fluids (liquids and gasses) can pass through the material unhindered.

Various filter materials were tested for gross porosity.

| | Thickness/ | | | |
|---|---|---|---|---|
| Material | microns | Time/s | GP | TAGP |
| Sachet paper | 140 | 53.3 | 0.019 | 0.134 |
| Viva ® Paper Towel | 560 | 0.5 | 2.000 | 3.571 |
| Blotting paper | 240 | 0.5 | 2.000 | 8.333 |
| Reynold's “Cut-Rite” wax | 20 | 540 | 0.002 | 0.093 |
| paper | | | | |
| Non-woven PP | 250 | 0.25 | 4.000 | 16.000 |
| Card Stock | 300 | 894 | 0.001 | 0.004 |
| Tyvek ® man-made fiber | 130 | 1320 | 0.001 | 0.006 |
| sheets | | | | |
| Photocopy paper | 80 | 59 | 0.017 | 0.212 |
| Silk Charmeuse | 150 | 0.5 | 2.000 | 13.333 |
| Knitted microfiber PET | 290 | 0.5 | 2.000 | 6.897 |

Damage to surfaces may include dulling of gloss, whitening or fogging or increased opacity of varnished surfaces, making finished surfaces sticky to touch, the transfer of print from the sachet decoration to the surface, peeling, cracking, blistering, and delamination of the surface. While not wishing to be limited to theory, it seems likely that the fragrance oil diffuses into the surface finish polymer matrix, and initially either plasticizes or partially dissolves it, causing dimensional changes such as swelling, as well as surface light scattering changes. Further softening of the finish can pick up print and adhere the sachet to the finish, leaving torn paper on the finish if the sachet is removed.

Some materials are better at containing the fragrance than others. For instance, poly(ethyleneterephthalate) (PET or PETE), high density poly(ethylene) (HDPE) and polypropylene (PP), including Ziegler-Natta catalyst and metallocene polymerized poly(olefins) including certain grades of poly(ethylene) (PE) and polypropylene (PP), mechanically treated PP such as oriented poly(propylene) (OPP), aluminum foil, metal shim, papers and films coated with UV cured crosslinked materials, poly(amides), such as nylon-6, and nylon-6,6, certain thermosetting resins, such as alkyd resins, polyester resins, epoxy resins, urea formaldehyde resins, melamine formaldehyde resins. The barrier film may also be a laminated composite of more than one type of film.

Some materials are too porous, and allow the diffusion of fragrance too fast, exhausting the fragrance too soon, and leading to a reduced product lifetime.

Mar Resistance Sheet Material Test—Varnished Wood:

Planed red oak planks of 3″×1″×24″ (available from Lowes' hardware store) were stained using Minwax Ebony 2718 penetrating stain (Minwax company NJ) applied with a paint brush. After allowing several hours to dry, a second coat was applied. This was allowed to soak in for 5 minutes before wiping off the excess with a paper towel. After 12 hours, coats of varnish were applied.

Minwax Fast Drying Polyurethane Clear Gloss varnish was applied to one of the wood surfaces. Four additional coats of the varnish were added, waiting at least 4 hours between applications.

Minwax Polycrylic Varnish was applied to a second stained plank in a similar manner.

Two coats of Behr Premium Plus Interior Ultra Pure White semi-gloss enamel was applied to another plank.

Samples of fragrance paper envelope sachets (comprising paper envelopes that are glue-sealed enclosures holding scent-impregnated materials) were placed on top of the finished wood for 5 hours. Another such paper sachet was placed on top of a CD jewel case. Properties for the paper for such paper envelope sachets may be nominally described as follows:

| Property | Average |
|---|---|
| Basis Weight | 69 |
| Caliper | 5.3 |
| Porosity | 19 |
| MD Tear | 61 |
| CD Tear | 64 |
| MD Tensile | 33.5 |
| CD Tensile | 16.4 |
| MD Stiffness | 3.5 |
| CD Stiffness | 1.9 |

| Surface | Results after 5 hours |
|---|---|
| Polycrylic varnish | Damaged - surface became so sticky that paper |
| | fragments were left behind when the sachet |
| | was removed. |
| Polyurethane varnish | No impact on this finish |
| White Paint | Surface became sticky. The sachet made a noise |
| | when taken off of the surface, however no paper |
| | or ink was transferred in 5 hours. |
| CD jewel case | Slight clouding |
| (PMMA) | |
| Rust-oleum Crystal | Stuck to the paper of sachets to the extent that |
| Clear Enamel spray | paper fragments were torn off when the |
| varnish | product was removed |
| Rust-oleum Painter's | Stuck to the paper of sachets to the extent that |
| Touch 2X Ultracover | paper fragments were torn off when the product |
| Clear Gloss | was removed |

Fragrance sachets prepared in accordance with presently disclosed subject matter were also placed onto the treated wood. These exemplary embodiment fragrance sachets were made from two differing sheet materials fused or bonded together to make a sachet. One side of the sachet is formed from a porous paper material, while the other is formed from a polymer such as PET, HDPE, PP, OPP, and/or a metalized film composite.

The sample filter paper for this exemplary embodiment as used in the tests herein comprised HEATSEALING FILTERPAPER121/2 from Glatfelter Scaër SAS (France). At Test Conditions: 23° C./50% rel. Humidity, the supplier indicates the following test specifications:

| Test-Name | Unit | Test-Method | Average |
|---|---|---|---|
| Basis Weight | g/m² | ISO 536 | 21.7 |
| Thickness | μm | ISO 534 | 69.9 |
| Tensile Strength Dry MD | N/15 mm | ISO 1924-2 | 19.1 |
| Tensile Strength Dry CD | N/15 mm | ISO 1924-2 | 6.3 |
| Tensile Strength Wet CD | N/15 mm | ISO 3781 | 2.1 |
| Brightness | % | ISO 2470-2 | 73.4 |
| Air permeability Akustron | l/m² · s | GLT | 681.5 |

The sample film for this exemplary embodiment as used in the tests herein comprised BF 48 HS/LS from Berry Plastics Corporation (Evansville, Ind.). Product description from the supplier describes the product as PET/PE/Foil/PE/Film, and indicates the following test specifications:

| | Typical | Unit of | Test |
|---|---|---|---|
| Test | Value | Measure | Method |
| Caliper | 3.99 | mil +/− 10% | |
| Tensile (MD) | 20.3 | lbs/in | ASTM 882 |
| Tensile (CD) | 20.7 | lbs/in | ASTM 882 |
| Tear (MD) | 202.7 | grams | ASTM 689 |
| Tear (CD) | 277.3 | grams | ASTM 689 |
| WVTR* | 0.0005 | g/100 sq in./24 hr | ASTM F1249 |
| O2TR* | 0.0005 | cc/100 sq in/24 hrs | ASTM D3985 |
| Puncture | 11.3 | lbs | FED TM2065 |
| Seals | 16.0 | lbs/in | ASTM F-88 |
| Curl | Pass | | |
| CoF | High Slip <0.20 | | |
| CoF | Low Slip 0.3-0.5 | | |

*Calculated upper limit from supplier data. Mocon measurements were conducted to validate but were limited to the instrument detection limit of 0.0023 g/100 in²/day for WVTR and 0.003 cc/100 in²/day for OTR.

The sachets were placed onto the finished wood, but alternately between film side down, and paper side down. Test results were as follows:

| | | |
|---|---|---|
| Surface | Results: paper side down, | Results: film side |
| | 24 hrs contact | down, 24 hrs contact |
| Polycrylic varnish | Damaged - significant | No impact |
| | amounts of paper fragments | |
| | and ink were left behind | |
| | when the sachet was | |
| | removed. | |
| Polyurethane | No impact on this finish | No impact |
| varnish | | |
| White Paint | Ink and paper transferred to | No impact |
| | the surface | |
| Compact Disk (CD) | Definite clouding and some | No impact |
| jewel case (PMMA) | transfer of paper marks | |

Another exemplary film which may be used with exemplary embodiments of presently disclosed subject matter comprises RP7xxWB from Sealed Air (Charlotte, N.C.). Product description from the supplier describes the product as a laminated Rollstock with a PET skin and a White PE metallocene sealant, and indicates the following specifications:

APPLICATION PROPERTIES

| | |
|---|---|
| Heat Seal Range: | 120-180° C. (248-356° F.) |
| Nominal Seal Strength: | 8 lbs/linear inch¹ |
| Core Diameter: | 76 or 152 mm (3 or 6 inch) |
| Storage Conditions (Rolls): | 27° C., 80% R.H. maximum, 18 months |

PERMEABILITY PROPERTIES OF AVAILABLE GAUGES
(Typical Values)

| | | OTR @ 23° C., | | MVTR @ 38° C., | |
|---|---|---|---|---|---|
| | | 0% R.H. | | 100% R.H. | |
| | Gauge | | cc/ | | g/ |
| Structure | (mils) | cc/m²/day | 100 in²/day | g/m²/day | 100 in²/day |
| RP720WB | 2.0 | 2.8 | .19 | 8.1 | .52 |
| RP725WB | 2.5 | 2.9 | .19 | 8.2 | .53 |
| RP730WB | 3.0 | 2.5 | .16 | 7.0 | .45 |
| RP735WB | 3.5 | 2.0 | .13 | 6.5 | .42 |
| RP740WB | 4.0 | 1.7 | .11 | 5.7 | .37 |
| RP745WB | 4.5 | 1.6 | .10 | 5.0 | .32 |
| RP750WB | 5.0 | 1.4 | .09 | 4.5 | .29 |

| | | |
|---|---|---|
| ASTM Method | D-3985 | F-1249 |

¹Typical values obtained from packaging equipment. Actual values will vary depending on equipment used and its operating conditions.

Mar Resistance on Cast Poly(Methylmethacrylate) (PMMA) Acrylic or Perspex Brand Man-Made PMMA Materials Sheets:

As the Compact Disk (CD) jewel cases were made from PMMA, a test was devised using a ¼″ cast PMMA sheet.

The thickness of a sample of the sheet material is measured using a micrometer. The sheet is cut into a circle of 66-70 mm diameter. This corresponds to the cap lid of Ball 4 oz Jelly Jar (Hearthmark LLC dba Jardin Home Brands, Fishers Ind.) with a standard mouth. These jars have a two-part lid. An essentially flat round metal surface that seals against the top of the wall of the glass jar, and a threaded collar, which compresses the lid against the glass jar mouth and locks the lid in place.

Fragranced vermiculite (the scent-impregnated materials in the above-referenced samples of fragrance paper sachets comprising paper envelopes that are glue-sealed enclosures) was stored in a Ball mason jar. 10 g+/−0.20 g was weighed into a Ball 4 oz Jelly Jar using a top pan chemical balance. The metal disk part of the lid was discarded and replaced with the paper, fabric, foil, or film to be tested. The screw collar was then used to lock the circular cut material over the mouth of the jar.

Each jar was then inverted and placed mouth-down onto the ¼″ thick clear cast poly(methylmethacrylate) (PMMA) (available from hardware retailers such as The Home Depot), and the time was noted. The jar was lifted off of the sheet periodically and the area in contact with the portion of the sheet visible through the collar is assessed for damage. In this test, 22 different materials were tested.

After 16 hours, some noticeable clouding occurred under all of the woven and non-woven fabric materials, including beneath Tyvek® man-made fiber sheets. Clouding was also noticed beneath 130 micron thick low density poly(ethylene) (LDPE) and Reynold's Wax Paper.

Similar results were noted at 24 and 72 hours. As progress of damage was slow for most of the samples, the sheet was carefully moved outside and placed in a tote in the Atlanta Ga. afternoon sun, along with a maximum/minimum thermometer to record the high and low temperature. The samples were examined after a total of 8 days, 5 of which were in a temperature range of between 67° F. and 125° F. Sometimes the acrylic sheet was damaged, in some cases the material being tested was damaged in some way, and in other cases, both were damaged. The damage to the acrylic sheet was to change the way that light interacts with the area exposed to the fragrance, as if the refractive index or a dimension of the surface layer was changed. This had the effect that you could see where the replaced lid of the jar was placed on the sheet.

| | | | Damage to |
|---|---|---|---|
| | | Damage to | barrier material |
| | Thickness/ | cast acrylic | being tested |
| Material | micron | sheet Y/N | Y/N |
| fragranced paper envelope | 140 | Y | N |
| sachets paper | | | |
| film sample from exemplary | 130 | N | N |
| embodiment of presently | | | |
| disclosed subject matter | | | |
| Blotting Paper | 240 | Y | N - but fibers |
| | | | stuck to |
| | | | acrylic |
| Viva ® Paper Towel | 560 | Y | N - but fibers |
| | | | stuck to |
| | | | acrylic |
| Silk charmuese | 150 | Y | Y discoloration |
| | | | Strongly |
| | | | adhered |
| Poly(propylene) non-woven | 250 | Y | N |
| PET knitted fabric | 290 | Y | N |
| Manilla file folder card stock | 300 | Y | N |
| Tyvek ® man-made | 130 | Y | N |
| fiber sheets | | | |
| Metalized food packaging | 130 | N | N |
| film (dog food bag) | | | |
| Aluminum foil | 10 | N | N |
| Reynold's Wax Paper | 20 | Y | N |
| High Density Poly(ethylene) | 400 | N | N |
| (HDPE) | | | |
| LDPE | 760 | N | N |
| LDPE | 130 | Y | N |
| Poly(ethylene terephthalate) | 340 | N | N |
| (PET) | | | |
| Poly(propylene) (PP) | 370 | N | N |
| Poly(vinyl chloride) (PVC) | 370 | N | Y- softened |
| | | | slightly |
| Poly(styrene) (PS) | 370 | N | Y- melted |
| | | | into acrylic |
| Cork PE coated paper | 110 | — | Y - sticky |
| Sachet paper, varnished with | 150 | Y | Y - sticky |
| Minwax polyurethane varnish | | | |
| PP | 900 | N | N |
| HDPE | 720 | N | N |

The nature of the damage to the cast acrylic sheet was interesting, as it did not correlate to the damage seen when sachets were placed on a CD jewel case, which turned cloudy. The two types of materials were assessed using crossed polarizing filters. Examination of the cast acrylic sheet used for the tests showed no stress marks were observed. However, examination of the plastics using crossed polarizing filters revealed multiple stress and flow marks in the CD jewel case, most likely due to the molding and/or extrusion during manufacturing.

As the process for damaging the acrylic was taking too long, we elected to add additional essential oil to the vermiculite. 224 g of fragranced vermiculite from the sample fragranced paper envelope sachet was thoroughly mixed with 22.4 g of cinnamon oil (Now Essential oils, Bloomington Ill.) and 22.4 g of clove oil (Now Essential Oils). 12 g of this mixture was placed into 4 oz mason jars. A sheet of the material to be tested is cut into a circle of 66-70 mm diameter. This corresponds to the cap lid of Ball 4 oz Jelly Jar (Hearthmark LLC dba Jardin Home Brands, Fishers Ind.) with a standard mouth. These jars have a two-part lid. An essentially flat round metal surface that seals against the top of the wall of the glass jar, and a threaded collar, which compresses the lid against the glass jar mouth and locks the lid in place. The cut circular material to be tested was placed inside the metal band, and screwed onto the jar containing the fragranced vermiculite.

CD jewel cases were disassembled, and the flat portions—the lid and the back—were used for tests. The jars were inverted and placed onto the jewel case lids and back. The jars were not disturbed for 5 days in an air conditioned room. The minimum temperature reached was 68° F./20° C. and the max was 76° F./29° C. The jars were carefully lifted off the acrylic and carefully examined.

One measure of damage to the sheet is to assess the turbidity (“whiteness”) of the sheet, which may be assessed by comparing the marred area with McFarland Turbity Standards contained in plastic cylindrical tubes. McFarland turbidity standards are sealed tubes of liquids containing a certain dilution of a latex, which makes the formulation turbid. The higher the concentration of the latex, the more turbid the liquid. They are recommended for estimating the concentration of bacteria in a liquid culture. The CD jewel cases were carefully examined in a shadow—that is to say, light impinged on the acrylic sheet from a low angle (from the side) while the turbidity was assessed from above. The turbidity of the PMMA jewel CD case was compared to the turbidity of the liquids in the McFarland tubes when viewed from the side of the sealed tubes and noted. The standards are compared by laying a sample tube on its side next to the marred area. Looking from above, through the center of the side wall of the tube in an area away from air bubbles, to the observer estimates whether the marring is worse or better than the standard selected. (McFarland 0.5, 1, 2, 3.) Other tubes are also compared until the one closest in turbidity to the damaged surface is identified.

Subject to specific test results for specific materials, packaging paper and similar materials may be used to thermally and ultrasonically bond to thermoplastics.

The damage level is recorded and then the inverted jar is replaced in the same spot after the assessment, so that damage can continue to accrue.

| | Thickness/ | McFarland | Damage to barrier |
|---|---|---|---|
| Material | micron | turbidity scale | material being tested Y/N |
| Tyvek ® man-made fiber | 130 | 1-2 | N |
| sheets | | | |
| fragranced paper envelope | 140 | 2-3 | N |
| sachets paper | | | |
| film sample from exemplary | 130 | 0 | N |
| embodiment of presently | | | |
| disclosed subject matter | | | |
| Blotting Paper | 240 | 0.5-1 (difficult | fibers stuck to |
| | | to assess | acrylic |
| | | because of | |
| | | fibers) | |
| Viva ® Paper Towel | 560 | 1-3 (difficult to | fibers stuck to |
| | | assess because | acrylic |
| | | of fibers) | |
| Silk charmuese | 150 | 2-3 | Y discoloration |
| | | | Strongly adhered |
| Manilla file folder card stock | 300 | 0.5-1 | N |
| Metalized food packaging film | 130 | 0 | N |
| (dog food bag) | | | |
| Aluminum foil | 10 | 0 | N |
| Reynold's Wax Paper | 20 | 1-2 | N |
| HDPE | 400 | 0 | N |
| LDPE | 760 | 0 | N |
| Cork PE coated paper | 110 | 2 | Y - sticky |
| LDPE | 130 | 0.5-2 | N |
| PET | 340 | 0 | N |
| Poly(propylene) (PP) | 370 | 0 | N |
| Poly(vinyl chloride) (PVC) | 370 | 0 | Y - softened slightly |
| Poly(styrene) (PS) | 370 | 0 - 0.5 acrylic | Y - melted into acrylic |
| | | appeared locally | |
| | | melted | |
| HDPE | 720 | 0 | N |

Direct Contact of Fragrance Oils onto Plastics.

1 drop of cinnamon oil was dropped onto various sheet plastic samples to assess damage due to fragrance oils and placed into plastic bags for 4 days. The same process was used to also test one drop of clove oil on the same plastics. The droplet initial contact angle was observed visually, but not measured. In the case of plastics thought to be likely to be attacked, the contact angle appeared to be very low indeed, compared to for instance HDPE.

| | |
|---|---|
| Cinnamon Oil - Low contact angle | PVC, PS, acrylic, Cork Industries |
| | coated paper, PET, metalized pet |
| | food bag. |
| Cinnamon Oil - High contact angle | HDPE, PP |
| Clove oil - Low contact angle | PVC, PS, acrylic, cork industries |
| | coated paper, metalized pet |
| | food bag. |
| Clove Oil - High contact angle | PET, HDPE, PP |

The materials were assessed for damage caused by the liquid oils after 4 days at room temperature:

| Plastic | Effect |
|---|---|
| PET - drinking water bottle | None (2 repetitions) |
| PET - hot bar food container | Both oils turned this plastic white |
| | (2 repetitions) |
| PET - Fruit packaging | Both oils turned this plastic white |
| PP | None |
| Metallized food packaging. | None |
| PMMA jewel case | Dissolved the plastic. |
| PS | Dissolved the plastic, leaving holes |
| PVC | PVC showed surface damage. |
| HDPE | None |
| water based PE coated | Oils eventually penetrated the barrier film, |
| paper, available from Cork | to make a stain on the underlying paper, |
| Industries (FL) | however no other visible damage to the |
| | composite occurred |

These data suggest that not all grades of PET are resistant to fragrance oils. Also, these data suggest several materials that are effective barriers to essential oils as well as some that are definitely not.

Materials that protect proximal or contiguous surfaces from fragrant oil in the sachet may be incorporated into the presently disclosed sachet structure in many different ways. For instance, if an exemplary embodiment of the presently disclosed fragrance sachet is intended to be free-standing, then the protective or barrier material could be formed into the shape of a receptacle (for example, a cup) in which the porous paper sachet sits. This embodiment can be rested upright on a surface without causing damage. If an exemplary embodiment of the presently disclosed sachet is to be placed on a polished surface, then the side that is intended to contact the surface may comprise the barrier material, while the porous material faces away from the surface.

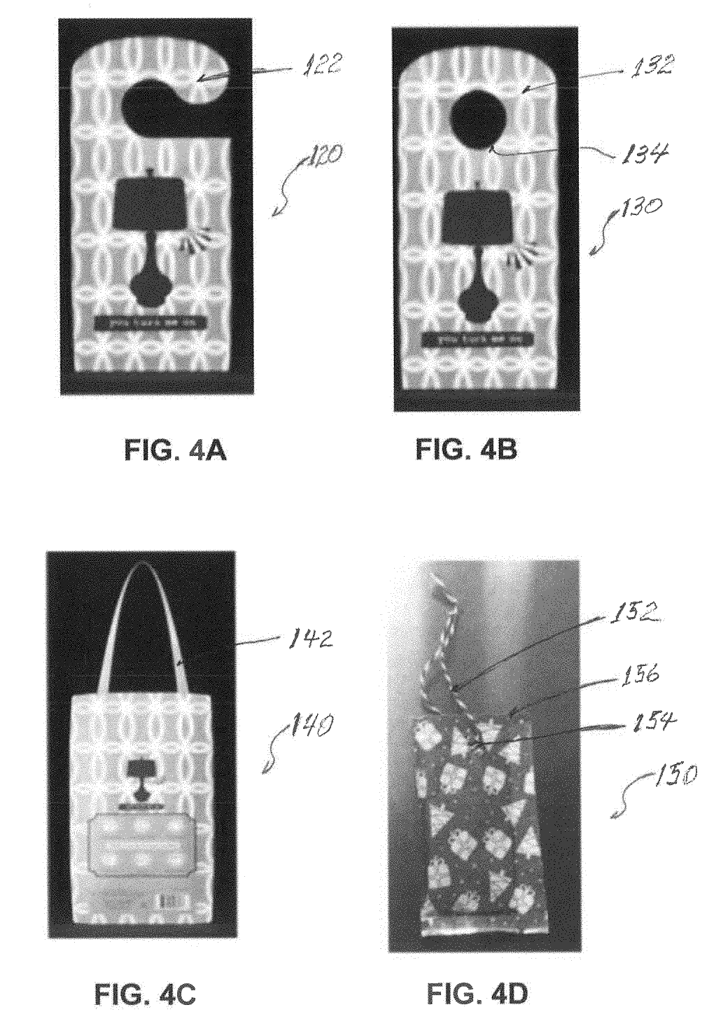

FIGS. 4A through 4D provide respective front perspective views of four additional exemplary embodiments of the presently disclosed subject matter, with respective integrated or associated attachment or mounting/hanging features or means.

More specifically, FIGS. 4A through 4D respectively illustrate further exemplary alternative fragrance sachet configurations 120, 130, 140, and 150. Each of the respective sachets has respective mounting or attachment means, such as features 122, 132, 142, and 152, respectively. Each of features 122, 132, and 142 may be, for example, integrally formed with the body of their respective sachets, or otherwise attached to the body thereof.

Feature 122 may comprise a general hook-shaped feature for engaging a surface or associated use location for securing its respective sachet 120. Feature 132 provides a formed opening, such as a circle or other shape, whereby such opening engages an intended surface or location for securing its respective sachet 130. Feature 142 may comprise a ribbon or other loop element attached to the body of it respective sachet 140, for engaging an intended surface or location.

Alternatively, a modification such as grommet 154 may be included in the design. In this way, the sachet embodiment 150 may for example be hung via a cord or ribbon 152 from a hanger, a hook, or a rear-view auto mirror, without directly contacting any finished surface. Such grommet 154 could be made from metal, or it could be made from a resilient plastic material. The grommet could be attached through a hole in the entire sachet that was cut separately, or it could be attached in a manner that stamps out the hole at the same time as fixing the grommet in place. Furthermore, a top seal bar such as at 156 may be incorporated into various of the presently disclosed exemplary embodiments in order to strengthen material used against ripping or tearing.

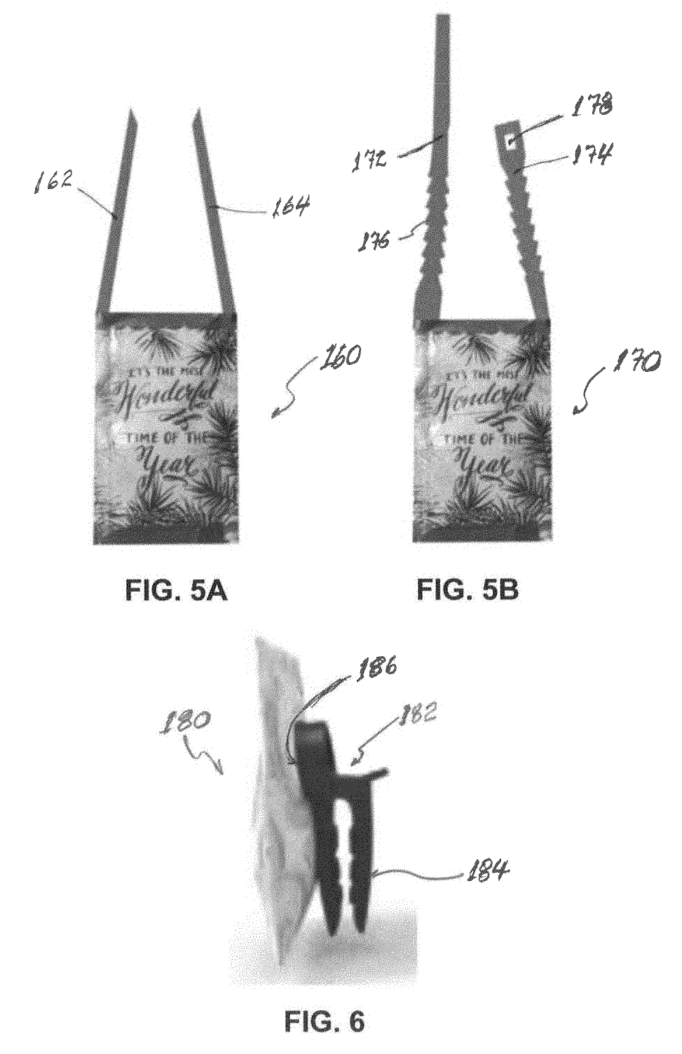

FIGS. 5A and 5B provide respective front perspective views of two additional exemplary fragrance sachet embodiments 160 and 170, respectively, of the presently disclosed subject matter, with respective integrated or associated attachment or mounting/hanging features or means.

In particular, FIG. 5A illustrates respective exemplary elements 162 and 164 projecting from the body of sachet 160. Such elements 162 and 164 may have respective mechanisms for attaching to each other, such as selective attachment by respective hook and latch type elements. Alternatively, they may have glue or other features, such as snaps for attaching to each other. Still further, elements 162 and 164 may simply be selective tied together in order to affix sachet 160 to or at a support feature at a desired location. As such, elements 162 and 163 may various comprise ribbon, or paper, or fabric, or other suitable materials, either integrally formed with or attached to the body of sachet 160.

FIG. 5B illustrates respective exemplary elements 172 and 174 projecting from the body of sachet 170. Such elements 172 and 174 may have respective mechanisms for attaching to each other, such as teeth or notches 176 which interact with and engage opening 178, in a zip-tie type of arrangement. Such exemplary attachment means permits a relatively stronger level of engagement between sachet 170 and an associated support surface or feature than some embodiments which hang or otherwise drape relative to their respective supports. Elements 172 and 174 may generally comprise relatively more rigid materials, to facilitate their respective zip-tie type engagement, such as plastics or other materials. Another example of a sufficiently rigidified material may comprise a composite of two or more materials, such as selected foil and filter materials which have been heat sealed together, along with die cut formation, to form together a relatively more rigid body for such exemplary zip-tie type functionality.

Sufficient rigidity and desired strength can be related in the context of composite embodiments. For example, Reynold's wax paper has demonstrable water porosity and gross porosity that would tend to indicate sufficient diffusion properties for an exemplary embodiment, but general strength of a sheet of such material may be less than desired for given embodiments. Those of ordinary skill in the art will appreciate that the strength of both paper and/or film features of any given exemplary embodiment of the presently disclosed subject matter may be assessed in a variety of ways, to determine, for example, burst strength, ballistic strength, tear strength, wet tear strength, and others. Those of ordinary skill in the art will likewise understand and appreciate various tests that may be performed in making such assessments for any given embodiment.

It is also to be understood from the complete disclosure herewith that the strength of certain papers and films can be reinforced by using them together and/or making a composite through lamination. The presently disclosed subject matter is intended to fully encompass such composite variations as may be desired for particular target characteristics of particular exemplary embodiments. For example, silk charmeuse could be used in combination with wax paper to make a suitably strong but desirably performing diffuser, or non-woven materials could be laminated to wax paper or to a thinner filter paper to add desired strength. Another potential composite in accordance with presently disclosed subject matter is a nonwoven fabric called SMS (spunbond-meltblown-spunbond). The meltblown PP film has very good porosity to moisture, yet is a high barrier to liquids, viruses, and bacteria, although the meltblown portion of the fabric itself is relatively delicate with little strength. In such instance, a much more open, much stronger spun bond fabric may be laminated on either side to provide the strength needed for use. Such resulting fabrics are for example of the type used in single use medical gowns, medical drapes, and the like.

FIG. 6 provides a generally side edge view of another exemplary fragrance sachet embodiment 180 of the presently disclosed subject matter incorporating or associated with alternative attachment or mounting features or means.

In particular, FIG. 6 represents an associated attachment or mounting feature or means 182, having for example an integral clip 184. Such clip 184 may facilitate attachment or association of sachet 180 with various supports, such as on vehicle air vents or other locations. Clip 184 may in turn be attached in various ways to the body of sachet 180. For example, respective snap elements may be incorporated into the surface of sachet 180 and feature 182, or other means may be used. For example, paired magnets (not shown) may be respectively included in the pouch of sachet 180 and on feature 182, so that sachet 180 is magnetically secured to structure 182 at magnetic interface 186, which via clip 184 is in turn further secured to an associated support. Alternatively, such a dual attachment feature or means may be practiced with another form of attachment in place of clip 184 formed into element 182. For example, a suction cup feature may be practiced at the interface 186.

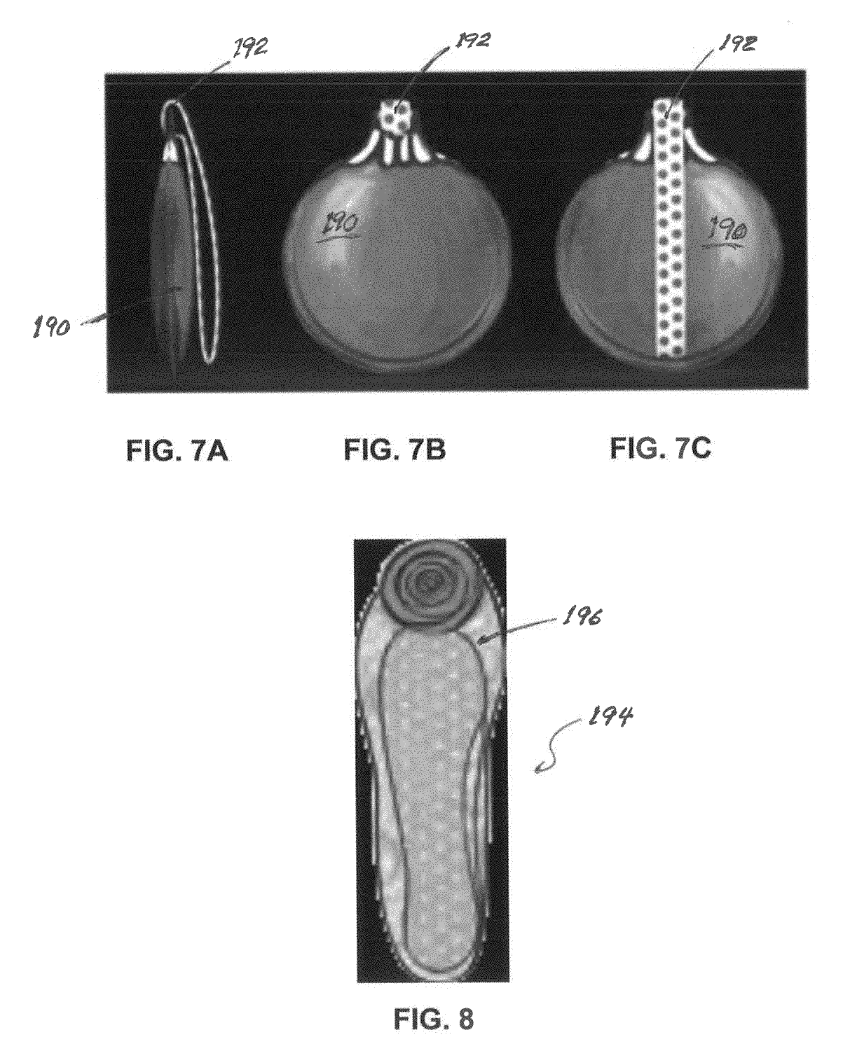

FIGS. 7A through 7C provide respective side edge, and opposing side views of another exemplary fragrance sachet embodiment 190 of the presently disclosed subject matter, incorporating an exemplary alternative resulting pouch shape.

In particular, a generally round pouch shape is provided, with color markings which potentially could be representative of fruit such as an apple or cherry, or which represented an object such as a seasonal ornament. The illustration represents that alternative shape and marking combinations may be practiced as desired, without departing from the general encompassing features of the presently disclosed fragrance sachet subject matter. Further, various attachment features or means may be practiced, as represented by exemplary loop 192. In addition, such exemplary loop 192 represents that the attachment features may themselves optionally be decorated or include other aesthetic and/or useful markings.

FIG. 8 is a front perspective view of another exemplary fragrance sachet embodiment 194 of the presently disclosed subject matter, illustrating another exemplary alternative resulting pouch shape.

In particular, as illustrated, the alternative embodiment of FIG. 8 has variations which are two-fold. First, the shape has been formed into the silhouette of a shoe, such as a slipper, and secondly, the markings 196 accompanying the sachet 194 also support the representation suggested by the silhouette. In other words, both the shape of the sachet 194 and the markings 196 thereon are coordinated to give the appearance of a slipper or shoe.

FIGS. 9A and 9B illustrate partially separated and partially assembled configurations, respectively, of a multiple-component pod-based exemplary fragrance sachet embodiment generally 200 of the presently disclosed subject matter, for alternative self-supporting features thereof.

In particular, the multi-component fragrance sachet generally 200 includes a supporting base structure generally 202, a pouch generally 204, and a pod or cover piece generally 206. Supporting base structure 202 may comprise, for example, a receptacle or cup-type shape formed from PET or PP materials. Alternative materials and shapes may be practiced, though a supportive, stable base which receives and/or effectively associates with the remaining components is preferred. For example, pouch 204 may comprise a component similar to the exemplary sachets illustrated in other figures as discussed herein, or may comprise a container formed from a single material, such as a filter-type material for passing fragrance therethrough from the enclosed content otherwise filling pouch 204. In some instances, such filter-type material may be the same or similar to coffee or teabag-type filter material.

The pod or cover piece generally 206 may comprise paper or some other breathable (i.e., fragrance permeable or filter-type) materials, to allow fragrance to be released from the sachet 200 as it passes from pouch 202. Further, pod 206 may include a coating on interior surfaces thereof, to facilitate its sealing or adhesion to base 202. Pod 206 may assume various shapes, but the provision of at least one tubular-shaped end 208 facilitates matching of such end with a circular shaped base. A relatively flattened end 210 generally results from sealing an end of a tubular construction. As referenced above, a string or similar element 212 may be provided for hanging of sachet 200. Those of ordinary skill in the art will understand from the complete disclosure herewith that alternative mounting or attachment elements or means may be used, as suggested herein.

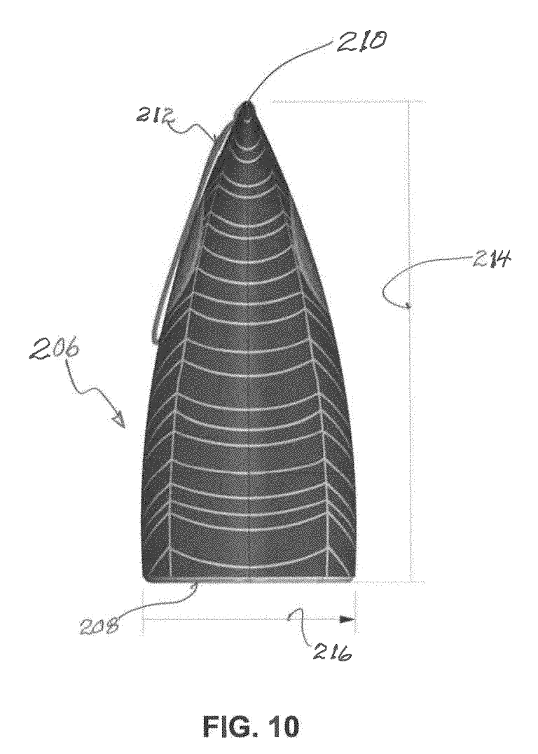

FIG. 10 is a generally side elevation view of an exemplary pod feature 206 of the presently disclosed subject matter for use in accordance with the exemplary multi-component fragrance sachet embodiment 200 represented in application FIGS. 9A and 9B.

In particular such pod 206 may assume various forms and shapes, but a generally elongated and relatively enlarged shape allows pod 206 to serve as a cover piece of a pouch component 204. With such generally elongated and enlarged shape, a resulting length (line 214) is typically longer than a resulting width (line 216). While various sizes may be practiced as desired, one exemplary size may have a length 214 of about 4 inches and a width 216 of about 1.75 inches. Also, the generally straight tubular portion adjacent the open end 208 may have a height of about 1 inch, in order to facilitate mating with a base 202 which may be provided of similar height.

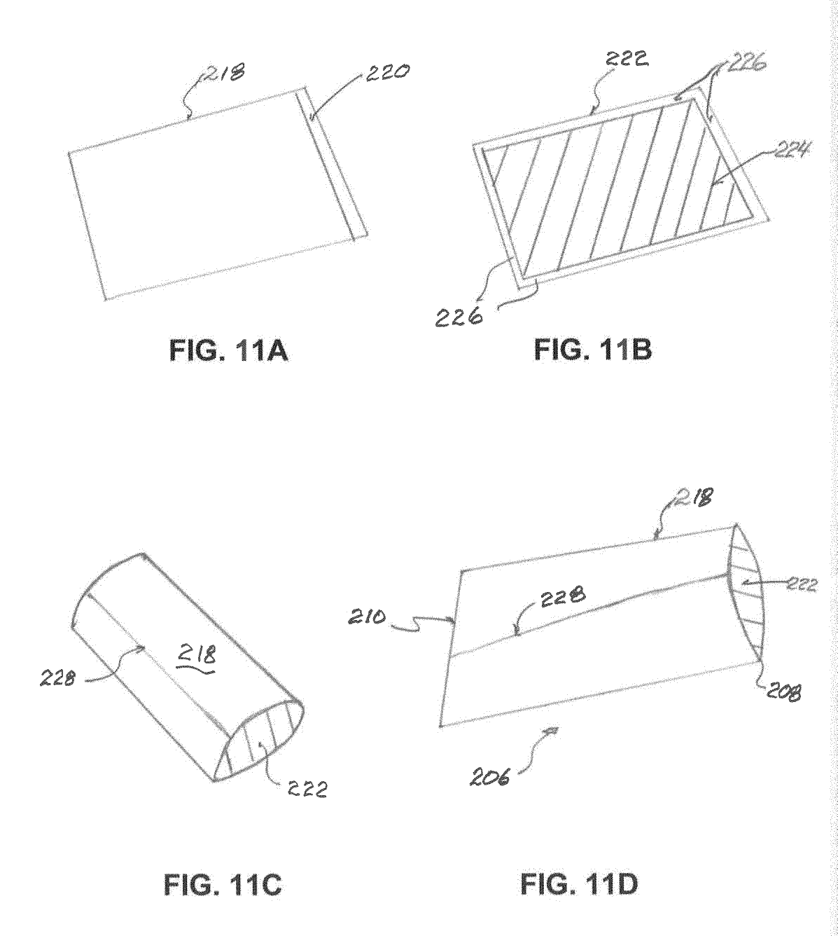

FIGS. 11A through 11D represent steps in an exemplary presently disclosed methodology for formation of a pod feature generally 206 for use in accordance with the exemplary embodiment represented in application FIGS. 9A and 9B.

In particular, FIG. 11A illustrates a first sheet of material generally 218 which may have an initial generally rectangular shape. For purposes of this example such sheet 218 may comprise an appropriately chosen paper product. The strip area or region 220 on paper sheet 218, situated along one edge thereof, may be treated to allow the paper to be more readily attached or sealed to a PP or PET material, if so used for base 202.

FIG. 11B illustrates a second sheet of material generally 222, which may also comprise a paper sheet or other material. Lining 224 indicated on such second sheet 222 is provided to show that the resulting exemplary pod 206 comprises two separate sheets. Sheet 222 may have a perimeter region generally 226 around the complete perimeter thereof, to serve similar to region 220 of sheet 218, as an area to be treated to allow the paper to be more readily attached or sealed to a PP or PET material, if so used for base 202.

As represented by FIG. 11C, first and second sheets 218 and 222, respectively, may be brought together to form a tube, with adjoining seam edges 228. Such joining may be accomplished through glue or other means of attachment well understood by those of ordinary skill in the art without further detailed description.

Once a tubular member is formed (FIG. 11C), one end 210 thereof may be sealed, as represented by FIG. 11D, while the opposite end 208 remains open. One exemplary embodiment of the pod or cover piece 206 is thus formed, for interaction with an associated base 202 and pouch 204, to form a fragrance sachet embodiment 200, as otherwise discussed herein. The PET or PP (or similar) base 202 of such resulting sachet 200 allows the sachet to be placed on a furniture surface by either end consumers or retailers without risk of damage to the supporting surface finish. Furthermore, use of a base component generally 202 permits both a retailer and an end user to be able to have sachet 200 stand alone on retail shelves, or in use. Also, the use of particularized finishes in areas 220 and 226 during construction of pod 206 can allow the sheets to be sealed to each other, without requiring separately applied adhesives. Placement of seal 228 along what amounts to one of the major surfaces of pod 206 also allows for a stronger construction.

It should be understood that the shapes and configurations of FIGS. 1 through 11D are provided by way of example only and may have any other suitable shape in alternative exemplary embodiments. For example, paired substrates 12 and 24 may have an oval shape, a star shape, a trefoil shape, a triangular shape, or a pentagonal shape in alternative exemplary embodiments. Similarly, any suitable graphic may be defined by pigment applied to the foil-based substrate. For example, a logo, at least one letter or series of letters, a word or series of words, a number or series of numbers, a geometric shape, and/or a picture may be printed on substrate 12 or 102 in alternative exemplary embodiments.

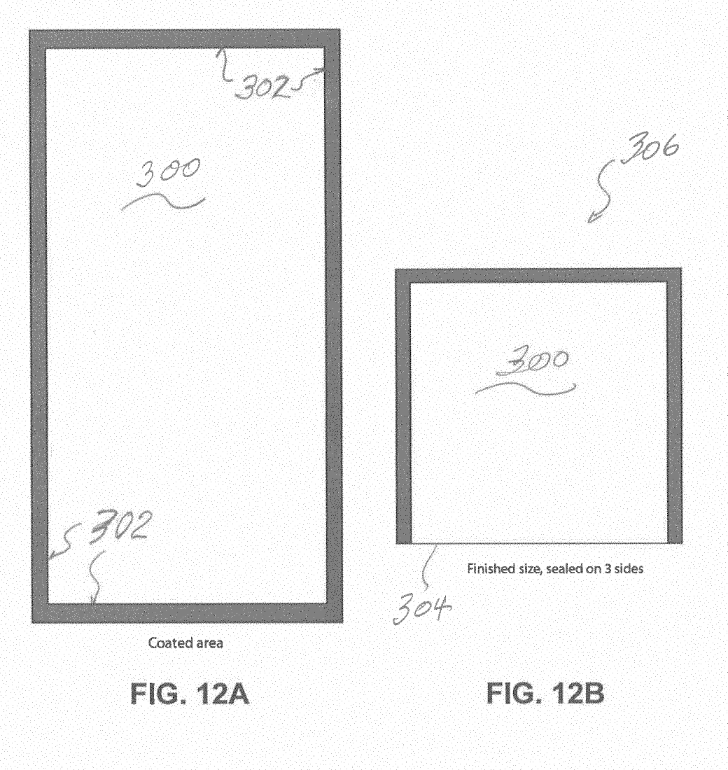

FIGS. 12A and 12B illustrate top elevation drawings representative of an exemplary paper coating process for a form/fill/seal paper sachet. Specifically, for example, a multi-layer component pouch 204 as used with the pod embodiment otherwise discussed herein may in some exemplary embodiments be replaced with a single sheet of material, such as paper. FIG. 12A represents a top elevation view of such an exemplary sheet of paper generally 300. As shown, such sheet may include coated peripheral regions 302 around the entirety thereof, which make respective contact whenever sheet 300 is folded about a symmetrical foldline 304 (see FIG. 12B). As shown, and as understood by those of ordinary skill in the art, once sheet 300 is so folded and is sealed at respective peripheral regions 302, a resulting pouch generally 306 is formed which may have been filled with fragrance evoking materials, as otherwise discussed herein. Also, such coatings at regions 302 may be of an adhesive nature for self-sticking, or may involve heat-sealable type materials, or represent some other sealing mechanism or area, such as crimping. A pouch 306 so formed may be used in combination with the pod-based embodiments otherwise discussed herein, or may be otherwise utilized, all in accordance with presently disclosed subject matter.

The present written description uses examples to disclose the presently disclosed subject matter, including the best mode, and also to enable any person skilled in the art to practice the presently disclosed subject matter, including making and using any devices or systems and performing any incorporated, involved, or corresponding methods. While the presently disclosed subject matter has been described in detail with respect to specific embodiments thereof, it will be appreciated that those skilled in the art, upon attaining an understanding of the foregoing, may readily adapt the present technology for alterations or additions to, variations of, and/or equivalents to such embodiments. Accordingly, the scope of the present disclosure is by way of example rather than by way of limitation, and the subject disclosure does not preclude inclusion of such modifications, variations, and/or additions to the presently disclosed subject matter as would be readily apparent to one of ordinary skill in the art.

What is claimed is:

1. An improved fragrance sachet, comprising:
a holder comprising a pouch formed of joined opposing sections, said joined opposing sections comprising respective different materials; and
a fragranced medium received within said pouch, for imparting a selected fragrance from said fragrance sachet.

2. An improved fragrance sachet as in claim 1, wherein one of said sections comprises a vapor impermeable material and the other of said sections comprises an at least partially vapor permeable material to facilitate fragrance dispersion.

3. An improved fragrance sachet as in claim 1, wherein said opposing sections comprise heat-sealable materials.

4. An improved fragrance sachet as in claim 1, wherein one of said sections comprises a foil-based substrate comprising a vapor impermeable material which supports the application of indicia thereon.

5. An improved fragrance sachet as in claim 1, wherein said other of said sections comprising an at least partially vapor permeable material supports the application of indicia thereon.

6. An improved fragrance sachet as in claim 1, wherein said opposing sections are joined through the use of at least one of heat-sealable materials, ultrasonic bonding, staples, separately applied adhesives, and crimping.

7. An improved fragrance sachet as in claim 1, wherein said pouch comprises one of a rectangular shape, a round shape, an oval shape, a star shape, a trefoil shape, a triangular shape, a pentagonal shape, and a preselected shape resembling an object.

8. An improved fragrance sachet as in claim 7, wherein at least one of said opposing sections comprises materials which support the application of indication thereon, coordinated at least in part with a preselected shape of said pouch resembling an object.

9. An improved fragrance sachet as in claim 7, wherein said pouch comprises a folded rectangular shape, with heat-sealable peripheral areas, to facilitate formation of said pouch using a form-fill-seal system.

10. An improved fragrance sachet as in claim 7, wherein each of said opposing sections comprise respective rectangles, with at least one of said sections comprising a contiguous thermoplastic film sheet, and the other of said sections comprising a paper layer with selected materials coating at least the perimeter of said paper layer.

11. An improved fragrance sachet as in claim 10, wherein said selected materials comprise at least one of water based latexes, PE emulsions, and PE dispersions printed onto said paper layer.

12. An improved fragrance sachet as in claim 2, wherein said vapor impermeable materials comprises at least one of metal foils, composites containing metal foils, metallized polymer films, polyimide films, polyethylene terephthalate (PET), high density poly(ethylene), poly(propylene) (PP), oriented poly(propylene) (OPP), poly(acrylonitrile), poly(amides) including nylon 6 and nylon 6,6, poly(vinyl fluoride) films, copolymers, a copolymer of (acrylonitrile) and (methyl acrylate) grafted with nitrile rubber, UV cured cross-linked films, and films and papers coated with UV cured coatings.

13. An improved fragrance sachet as in claim 1, wherein:
said fragranced medium comprises at least one of vermiculite, bulk absorbent material which may be fragranced, powders, volcanic rock, and pumice, treated with fragrance materials; and
said fragrance materials comprise at least one of naturally occurring fragrance oils, synthetic oils, manufactured blends of fragrance oils, and blends between natural and manufactured origin materials.

14. A fragrance sachet device for disseminating fragrance, without requiring power, comprising a holder for a medium treated with essential oils, said holder comprising a pouch formed with respective different at least first and second materials on opposing sides thereof, with one of said materials selected from a vapor impermeable material to protect a supporting surface against the ingress of essential oils, and the other of said materials selected from a filter type material to facilitate fragrance dispersion.

15. A fragrance sachet device as in claim 14, wherein said opposing side materials comprise heat-sealable materials.

16. A fragrance sachet device as in claim 14, wherein said filter type material comprises an at least partially vapor permeable material including at least one of a plant based paper, a synthetic paper, a non-woven material, a woven, perforated films, and a knitted material.

17. A fragrance sachet device as in claim 14, wherein said filter type material includes a coating that is sealable to another thermoplastic material.

18. A fragrance sachet device as in claim 14, further comprising attachment means associated with said holder, for attaching said holder to an associated supporting structure.

19. A fragrance sachet device as in claim 18, wherein said attachment means comprises hanging support for hanging said holder from an associated supporting structure.

20. A multiple-component, self-supporting, pod-based fragrance sachet, comprising:
a supporting base structure;
a pouch;
a fragranced medium received within said pouch; and
a pod cover piece for covering said pouch, and interfacing with said supporting base structure for support of said fragrance sachet.

21. A multiple-component, self-supporting, pod-based fragrance sachet as in claim 20, wherein said supporting base structure comprises a cup-type shaped receptacle, providing a supportive, stable base.

22. A multiple-component, self-supporting, pod-based fragrance sachet as in claim 21, wherein said supporting base structure is formed from one of PET and PP materials.

23. A multiple-component, self-supporting, pod-based fragrance sachet as in claim 20, wherein said pouch includes a filter-type material for passing fragrance therethrough from the enclosed content otherwise filling said pouch.

24. A multiple-component, self-supporting, pod-based fragrance sachet as in claim 20, wherein said pod cover piece comprises an at least partially fragrance permeable material.

25. A multiple-component, self-supporting, pod-based fragrance sachet as in claim 24, wherein said pod cover piece comprises paper having a coating on selected interior surfaces thereof, to facilitate sealing of said pod and adhesion to said supporting base structure.

26. A multiple-component, self-supporting, pod-based fragrance sachet as in claim 20, wherein said pod cover piece has at least one tubular-shaped end for matching with said cup-type shaped receptacle of said supporting base structure.

27. A multiple-component, self-supporting, pod-based fragrance sachet as in claim 26, wherein said pod cover piece has a flattened, sealed end thereof opposite to said at least one tubular-shaped end thereof.

28. A multiple-component, self-supporting, pod-based fragrance sachet as in claim 20, further including hanging means for hanging said fragrance sachet.

29. A multiple-component, self-supporting, pod-based fragrance sachet as in claim 20, wherein said pod cover piece has a height of less than about 5 inches, and a width of less than about 2 inches.

30. A multiple-component, self-supporting, pod-based fragrance sachet as in claim 20, wherein:
said supporting base structure comprises a cup-type shaped receptacle formed from one of PET and PP materials;
said pouch includes a filter-type material for passing fragrance therethrough from the enclosed content otherwise filling said pouch; and
said pod cover piece comprises at least partially fragrance permeable paper having a coating on selected interior surfaces thereof, to facilitate sealing of said pod, and said pod cover piece has at least one tubular-shaped end for matching with said cup-type shaped receptacle of said supporting base structure, and has a flattened, sealed end thereof opposite to said at least one tubular-shaped end thereof.

31. Methodology for producing a form/fill/seal fragrance sachet, comprising:
providing a single sheet of at least partially vapor permeable material;
treating peripheral regions around the entirety of said single sheet of material;
folding said single sheet of material about a symmetrical foldline so that respective edges make contact whenever said sheet is folded;
sealing at selected respective peripheral regions so that a resulting pouch is formed with an open mouth;
filling said pouch with a fragranced medium; and
sealing the open mouth of said resulting pouch, to provide a formed/filled/sealed fragrance sachet.

32. Methodology as in claim 31, wherein said single sheet of material comprises paper.

33. Methodology as in claim 31, wherein said treating includes providing adhesive materials at said respective peripheral regions for self-sticking when said sheet is folded, or providing heat-sealable type materials at said respective peripheral regions.

34. Methodology as in claim 31, wherein said treating includes selecting peripheral regions for sealing, and said sealing comprises mechanical sealing through crimping.

35. Methodology for providing a multiple-component, self-supporting, pod-based fragrance sachet, comprising:
providing a supporting base structure;
providing a pouch with a fragranced medium received within said pouch; and
providing a pod cover piece for covering said pouch, and interfacing with said supporting base structure for support of said fragrance sachet.

36. Methodology as in claim 35, wherein said pod cover piece comprises two joined sheets of material, folded to form a generally tubular shape with one end thereof sealed.

37. Methodology as in claim 35, wherein:
said supporting base structure comprises a cup-type shaped receptacle formed from one of PET and PP materials;
said pouch includes a filter-type material for passing fragrance therethrough from the enclosed fragranced medium otherwise received within said pouch; and
said pod cover piece comprises at least partially fragrance permeable paper having a coating on selected interior surfaces thereof, to facilitate sealing of said pod, and said pod cover piece has at least one tubular-shaped end for matching with said cup-type shaped receptacle of said supporting base structure, and has a flattened, sealed end thereof opposite to said at least one tubular-shaped end thereof.

38. Methodology as in claim 35, wherein:
said supporting base structure comprises a cup-type shaped receptacle, providing a supportive, stable base; and
said pod cover piece comprises an at least partially fragrance permeable material.

39. Methodology as in claim 35, further including hanging means for hanging said fragrance sachet.

40. Methodology for producing a fragrance sachet, comprising the steps of:
sealing a vapor impermeable-based substrate to a filter-based substrate to form a partially open pouch;
filling the pouch with scent-impregnated material; and
closing the pouch to form a fragrance sachet.

41. Methodology as in claim 40, further including providing a graphic presentation on the vapor impermeable-based substrate.

42. Methodology as in claim 40, wherein said substrates respectively comprise heat-sealable materials.

43. Methodology as in claim 40, wherein said scent-impregnated material comprises at least one of vermiculite, bulk absorbent material which may be fragranced, powders, volcanic rock, and pumice, treated with at least one of naturally occurring fragrance oils, synthetic oils, manufactured blends of fragrance oils, and blends between natural and manufactured origin materials.

44. Methodology as in claim 40, wherein said sealing step includes the use of at least one of heat-sealable materials, ultrasonic bonding, staples, separately applied adhesives, and crimping, for sealing said respective substrates.

45. Methodology as in claim 40, wherein said fragrance sachet comprises one of a rectangular shape, a round shape, an oval shape, a star shape, a trefoil shape, a triangular shape, a pentagonal shape, and a preselected shape resembling an object.

46. Methodology as in claim 40, wherein said pouch comprises a folded rectangular shape, with heat-sealable peripheral areas, to facilitate formation of said pouch using a form-fill-seal system.

47. Methodology as in claim 40, wherein each of said opposing substrates comprise respective rectangles, with at least one of said substrates comprising a contiguous thermoplastic film sheet, and the other of said substrates comprising a paper layer with selected materials coating at least the perimeter of said paper layer.

48. Methodology as in claim 47, wherein said selected materials comprise at least one of water based latexes, PE emulsions, and PE dispersions printed onto said paper layer.

49. Methodology as in claim 40, wherein said vapor impermeable-based substrate comprises at least one of metal foils, composites containing metal foils, metallized polymer films, polyimide films, polyethylene terephthalate (PET), high density poly(ethylene), poly(propylene) (PP), oriented poly(propylene) (OPP), poly(acrylonitrile), poly(amides) including nylon 6 and nylon 6,6, poly(vinyl fluoride) films, copolymers, a copolymer of (acrylonitrile) and (methyl acrylate) grafted with nitrile rubber, UV cured cross-linked films, and films and papers coated with UV cured coatings.
